(19) United States
(12) Patent Application Publication
(10) Pub. No.: US 2026/0294996 A1
(43) Pub. Date: Oct. 1, 2026

(54) COMPOSITIONS CONTAINING KRATOM COMPOUNDS

(71) Applicant: CAAMTECH, INC., Issaquah, WA (US)

(72) Inventor: Andrew R. CHADEAYNE, Issaquah, WA (US)

(73) Assignee: CAAMTECH, INC., Issaquah, WA (US)

(21) Appl. No.: 19/690,811

(22) Filed: May 28, 2026

Related U.S. Application Data
(62) Division of application No. 18/352,677, filed on Jul. 14, 2023.
Application No. 18/352,677 is a continuation of application No. 17/072,246, filed on Oct. 16, 2020, now abandoned.
(60) Provisional application No. 62/915,857, filed on Oct. 16, 2019.
(60) Provisional application No. 62/915,859, filed on Oct. 16, 2019.
(60) Provisional application No. 62/915,861, filed on Oct. 16, 2019.
(60) Provisional application No. 62/915,864, filed on Oct. 16, 2019.
(60) Provisional application No. 62/915,866, filed on Oct. 16, 2019.
(60) Provisional application No. 62/915,871, filed on Oct. 16, 2019.
(60) Provisional application No. 62/915,873, filed on Oct. 16, 2019.
(60) Provisional application No. 62/915,877, filed on Oct. 16, 2019.
(60) Provisional application No. 62/915,879, filed on Oct. 16, 2019.
(60) Provisional application No. 62/915,881, filed on Oct. 16, 2019.
(60) Provisional application No. 62/915,883, filed on Oct. 16, 2019.
(60) Provisional application No. 62/915,886, filed on Oct. 16, 2019.
(60) Provisional application No. 62/915,889, filed on Oct. 16, 2019.
(60) Provisional application No. 62/915,893, filed on Oct. 16, 2019.
(60) Provisional application No. 62/915,896, filed on Oct. 16, 2019.
(60) Provisional application No. 62/915,898, filed on Oct. 16, 2019.
(60) Provisional application No. 62/915,903, filed on Oct. 16, 2019.
(60) Provisional application No. 62/915,906, filed on Oct. 16, 2019.
(60) Provisional application No. 62/915,912, filed on Oct. 16, 2019.
(60) Provisional application No. 62/915,914, filed on Oct. 16, 2019.
(60) Provisional application No. 62/915,917, filed on Oct. 16, 2019.
(60) Provisional application No. 62/915,920, filed on Oct. 16, 2019.
(60) Provisional application No. 62/915,923, filed on Oct. 16, 2019.
(60) Provisional application No. 62/915,925, filed on Oct. 16, 2019.
(60) Provisional application No. 62/915,927, filed on Oct. 16, 2019.
(60) Provisional application No. 62/915,929, filed on Oct. 16, 2019.

(51) Int. Cl. A61K 36/74; A61K 45/06

(52) U.S. Cl. CPC A61K 36/74; A61K 45/06

(57) ABSTRACT

Disclosed are compositions containing (a) a purified kratom compound chosen from the following: 7-Hydroxymitragynine, Mitragynine, Paynantheine, Speciociliatine, Speciogynine, Ajmalicine, Ciliaphylline, Corynantheidine, Corynoxine A, Corynoxine B, Isomitraphylline, Isorhynchophylline, Mitraphylline, Rhynchophylline, Speciophylline, Speciofoline, Epicatechin, 7-Hydroxyspecioliatine, 9-Hydroxycorynantheidine, Corynoxeine, Isopteropodine, Isorhynchophylline Oxindole, Tetrahydroalstonine, Mitragynine Oxindole B, and Mitragynine Oxindole A or the salts of these kratom compounds; and (b) a second active compound selected from a serotonergic drug, a purified psilocybin derivative, a purified cannabinoid, or a purified terpene. The disclosure also relates to formulations, including pharmaceutical formulations, of such a composition and an excipient. Also disclosed are methods of regulating the activity of a neurotransmitter receptor and methods of treating a psychological disorder, a compulsive disorder, or a depressive disorder.

### CROSS-REFERENCE TO RELATED APPLICATIONS

This application is a Divisional application of U.S. application Ser. No. 18/352,677, filed Jul. 14, 2023; which is a Continuation application of U.S. application Ser. No. 17/072,246, filed on Oct. 16, 2020; which claims priority to U.S. Provisional Application Nos. 62/915,857; 62/915,859; 62/915,861; 62/915,864; 62/915,866; 62/915,871; 62/915,873; 62/915,877; 62/915,879; 62/915,881; 62/915,883; 62/915,886; 62/915,889; 62/915,893; 62/915,896; 62/915,898; 62/915,903; 62/915,906; 62/915,912; 62/915,914; 62/915,917; 62/915,920; 62/915,923; 62/915,925; 62/915,927; and 62/915,929; all filed Oct. 16, 2019, the disclosures of which are each incorporated by reference.

### TECHNICAL FIELD

The disclosure relates to new compositions and formulations of purified kratom compounds as well as methods for the therapeutic use of those compositions and formulations.

### BACKGROUND

The leaves of several genera of the plant Mitragynia contain many phytochemical compounds that are biologically active. One species, Mitragynia speciosa (scientific name Mitragynia speciosa [Korth.] Havil.) also known as kratom, is a tropical evergreen tree found in several Southeast Asian countries including Thailand, Indonesia, Papua New Guinea, Malaysia (where it is known as “biak baik”), and Myanmar.¹ For hundreds of years, kratom has been used by the local indigenous people in their traditional medicine practices. Kratom leaves are typically consumed directly by chewing, steeped for use as teas, decoctions, tinctures, and in cold-soaked macerations.¹

At low doses, kratom has stimulating effects and is often used by laborers to increase endurance and combat boredom.¹ At higher doses, kratom has opioid-like effects and, as a result, is used as a general analgesic.² Other biological effects of kratom include anti-cancer and antimicrobial effects. Kratom is also used as a substitute for opium or to treat opium withdrawal symptoms. It is also used by some for self-treatment for opioid withdrawal.³ Other medical applications of kratom include treating musculoskeletal pain,⁴ fever, cough, diarrhea, depression, and anxiety.⁵⁻⁹

Researchers have identified over 40 alkaloid and non-alkaloid compounds in kratom leaves including mitragynine, 7-hydroxymitragynine, epicatechin, ciliaphylline, paynantheine, and tetrahydroalstonine.^{1,2} Mitragynine, in particular, is considered the primary alkaloid constituent of kratom. Up to 66% by weight of crude alkaloid extracts from kratom consist of mitragynine.

Mitragynine was first isolated by Hooper in 1907.¹⁰ In 1921, Field replicated the work of Hooper and named the compound mitragynine.¹¹ The first crystal structure was solved in the 1960s by Zacharias, et al.¹² The other major alkaloids of kratom are paynantheine, speciogynine, and speciociliatine.¹³ The quantities of these major alkaloids vary greatly among different regions where the plants grow. Variations in the compounds present and their amounts are also dependent on the age of the plant, environmental, and soil conditions. These uncontrolled variables make it difficult to accurately interpret the reported psychoactive and medicinal effects of the raw plant material.^{5,8,13,14}

In 2016, Kruegel et al. used radioligand displacement assays to study the human mu opioid receptor (hMOR) binding affinity and functional activity of the major kratom alkaloids.¹⁵ The data indicated the Kᵢ values for mitragynine and 7-OH were 233 nM and 47 nM, respectively. Bioluminescence resonance energy transfer (BRET) functional assay data showed both compounds behaved as partial agonists at hMOR. However, 7-OH had a greater potency (EC₅₀=35 nM) compared to mitragynine (EC₅₀=339 nM). Three other major kratom alkaloids paynantheine, speciogynine, and speciociliatine had IC₅₀ values in the micromolar range. These compounds also exhibited competitive antagonist activity at hMOR. Mitragynine and 7-OH are also active at the human kappa and delta opioid receptors (hKOR and hDOR, respectively).¹⁵ Both compounds act as competitive antagonists at hKOR with IC₅₀ values in the micromolar range. At hDOR, however, both compounds exhibited weak antagonist activity. And, while binding was observed for 7-OH, binding was negligible for mitragynine at hDOR.

Among the minor alkaloids in kratom, the oxidized derivative 7-hydroxymitragynine (7-OH) is of particular interest to researchers. Studies indicate that this compound exhibits analgesic effects that are mediated through agonist activity at hMOR. The data show that the potency of 7-OH at hMOR exceeds that of the opioid agonist morphine. ^{16,17}

Mitragynine is also known to bind to non-opioid receptors, including alpha-2 adrenergic receptors, adenosine A_{2A} receptors, dopamine D₂ receptors, and serotonin 5-HT_{2C} and 5-HT₇ receptors.³ However, the strength of the binding affinities has not been established.

Several studies indicate that raw kratom extracts possess analgesic capabilities in rodents. ¹⁸⁻²² The main kratom component mitragynine also elicits analgesic effects in several species.^{6,16,18,21,23} Interestingly, the route of administration appears to be critical for the analgesic effects from mitragynine to occur. Mitragynine had an analgesic potency equivalent to codeine in mice, rats, and dogs when given intraperitoneally or orally.²³ However, mitragynine was inactive as an analgesic in mice and rats when administered subcutaneously. Researchers speculated that perhaps an active metabolite of mitragynine formed during the first-pass metabolism after oral or intraperitoneal administration was responsible for the analgesic effect. However, this hypothesis does not hold up based on a 1996 study by Matsumoto et al.¹⁶ In this study, it was observed that mitragynine is also an active analgesic using intracerebroventricular administration. Here, the first-pass metabolism would not be expected to play a significant role, yet the analgesic effect was still observed. Therefore, more research is required to understand kratom, its components, and their interactions.

From the promising results using kratom and single isolated compounds from it, there is an unmet need for formulated products providing specific combinations of purified kratom compounds from Mitragynia speciosa. For example, there exists a need for formulations wherein each purified kratom compound is separated from the plant leaf with which it coexisted in nature and purposefully recombined to optimize the effects on the subject to which it is administered.

### SUMMARY

The disclosure relates to compositions comprising, consisting essentially of, or consisting of (a) a purified kratom compound chosen from the following: 7-Hydroxymitragynine, Mitragynine, Paynantheine, Speciociliatine, Speciogynine, Ajmalicine, Ciliaphylline, Corynantheidine, Corynoxine A, Corynoxine B, Isomitraphylline (also known as Isomitraphylline Oxindole), Isorhynchophylline, Mitraphylline, Rhynchophylline, Speciophylline, Speciofoline, Epicatechin, 7-Hydroxyspecioliatine, 9-Hydroxycorynantheidine, Corynoxeine, Isopteropodine, Isorhynchophylline Oxindole, Tetrahydroalstonine, Mitragynine Oxindole B, and Mitragynine Oxindole A or the salts of these kratom compounds; and (b) a second active compound selected from a serotonergic drug, a purified psilocybin derivative, a purified cannabinoid, or a purified terpene. In a composition of the disclosure the molar ratio of the purified kratom compound to the second active compound in the composition is from 0.1:100 to 100:01, from 1:100 to 100:1, from 1:50 to 50:1, from 1:25 to 25:1, from 1:20 to 20:1, from 1:10 to 10:1, from 1:5 to 5:1, from 1:2 to 2:1 or is 1:1.

The disclosure also relates to formulations comprising a composition of comprising, consisting essentially of, or consisting of (a) a purified kratom compound selected from the following: 7-Hydroxymitragynine, Mitragynine, Paynantheine, Speciociliatine, Speciogynine, Ajmalicine, Ciliaphylline, Corynantheidine, Corynoxine A, Corynoxine B, Isomitraphylline, Isorhynchophylline, Mitraphylline, Rhynchophylline, Speciophylline, Speciofoline, Epicatechin, 7-Hydroxyspecioliatine, 9-Hydroxycorynantheidine, Corynoxeine, Isopteropodine, Isorhynchophylline Oxindole, Tetrahydroalstonine, Mitragynine Oxindole B, and Mitragynine Oxindole A or the salts of these kratom compounds; (b) a second active compound selected from a serotonergic drug, a purified psilocybin derivative, a purified cannabinoid, and a purified terpene; and (c) an excipient. The formulation may be a pharmaceutical formulation comprising, consisting essentially of or consisting of (a) a purified kratom compound, (b) a second active compound, and (c) a pharmaceutically acceptable excipient, where a purified kratom compound and the second active compound are each present in a therapeutically effective amount.

Also disclosed are methods of regulating the activity of a neurotransmitter receptor by administering to a person in need thereof an effective dose of a composition of the disclosure or of a formulation of the disclosure or by administering to a person in need thereof a pharmaceutical formulation of the disclosure.

The disclosure also provides methods of treating a psychological disorder, a compulsive disorder, or a depressive disorder by administering to a person in need thereof an effective dose of a composition of the disclosure or of a formulation of the disclosure or by administering to a person in need thereof a pharmaceutical formulation of the disclosure.

### DETAILED DESCRIPTION

Disclosed herein are new compositions and formulations of a purified kratom compound and a second active compound as well as methods for the therapeutic use of those compositions and formulations. The compositions comprise, consist essentially of, or consist of (a) a purified kratom compound chosen from the following: 7-Hydroxymitragynine, Mitragynine, Paynantheine, Speciociliatine, Speciogynine, Ajmalicine, Ciliaphylline, Corynantheidine, Corynoxine A, Corynoxine B, Isomitraphylline, Isorhynchophylline, Mitraphylline, Rhynchophylline, Speciophylline, Speciofoline, Epicatechin, 7-Hydroxyspecioliatine, 9-Hydroxycorynantheidine, Corynoxeine, Isopteropodine, Isorhynchophylline Oxindole, Tetrahydroalstonine, Mitragynine Oxindole B, and Mitragynine Oxindole A or the salts of these kratom compounds; and (b) a second active compound selected from a serotonergic drug, a purified psilocybin derivative, a purified cannabinoid, or a purified terpene. Exemplary molar ratios of the purified kratom compound to the second active compound in a composition of the disclosure include but are not limited to from about 0.1:100 to about 100:01, from about 1:100 to about 100:1, from about 1:50 to about 50:1, from about 1:25 to about 25:1, from about 1:20 to about 20:1, from about 1:10 to about 10:1, from about 1:5 to about 5:1, from about 1:2 to about 2:1 or may be about 1:1.

The compositions and formulations disclosed here do not occur in nature. In each the kratom compound and second active compound are combined into compositions and formulations via human ingenuity to arrive at compositions and formulations that are not found in nature. These compositions and formulations have different physical properties from how the compounds in them are found in nature and provide different pharmacological properties. In many cases, the disclosed compositions and formulations provide different clinical effects when administered to a subject. A composition of the disclosure may be prepared using techniques known in the art such as mixing of a purified kratom compound and a second active compound, forming a solution or slurry of a purified kratom compound and a second active compound followed by solvent removal and other such techniques.

The purified kratom compound may be chosen from the following: 7-Hydroxymitragynine, Mitragynine, Paynantheine, Speciociliatine, Speciogynine, Ajmalicine, Ciliaphylline, Corynantheidine, Corynoxine A, Corynoxine B, Isomitraphylline, Isorhynchophylline, Mitraphylline, Rhynchophylline, Speciophylline, Speciofoline, Epicatechin, 7-Hydroxyspecioliatine, 9-Hydroxycorynantheidine, Corynoxeine, Isopteropodine, Isorhynchophylline Oxindole, Tetrahydroalstonine, Mitragynine Oxindole B, and Mitragynine Oxindole A or the salts of these kratom compounds.

The structural formulas of each of these kratom compounds is shown below. Each is naturally present in many naturally occurring organisms, including Mitragyna speciosa, and occurs as a minor component within a mixture of many other molecules.

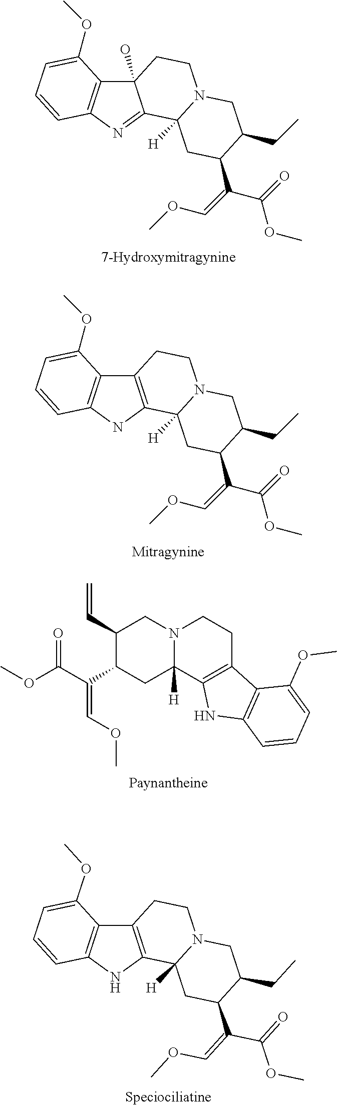

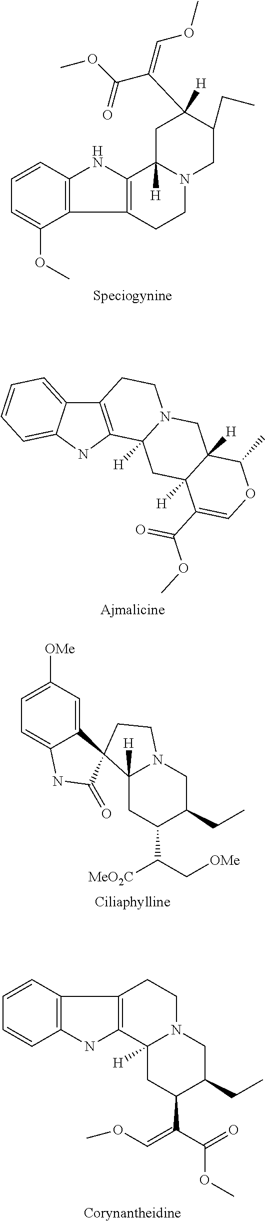

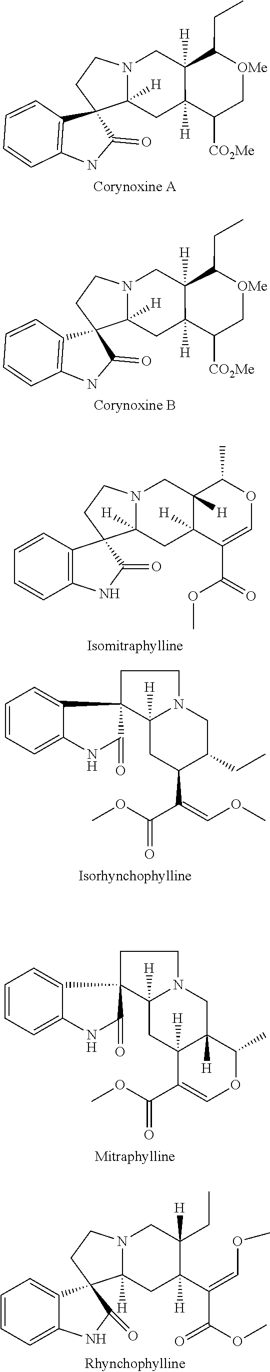

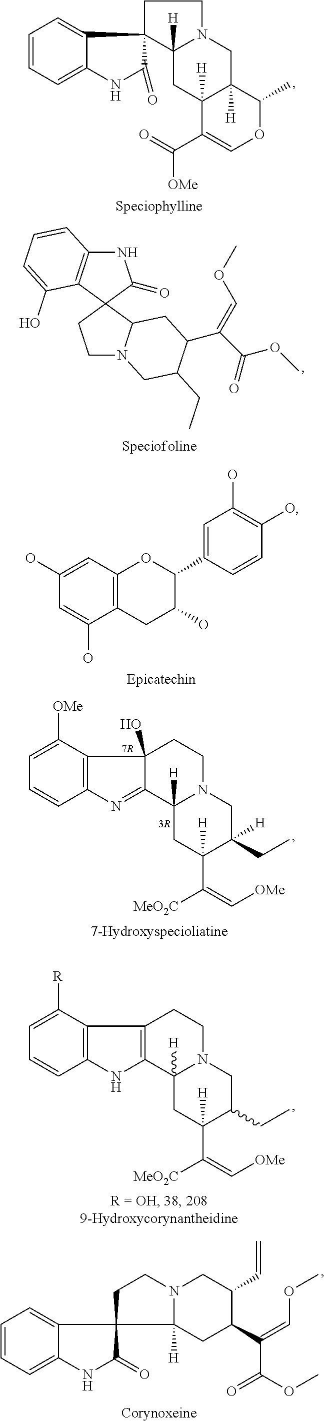

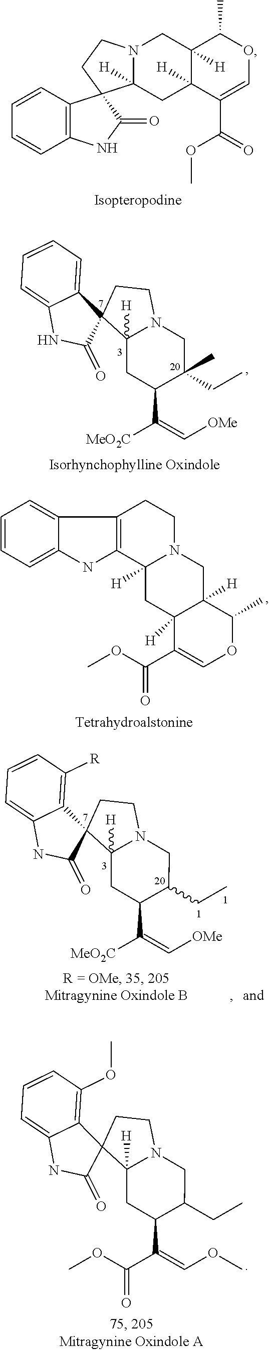

Each of these compounds is naturally present in many naturally occurring organisms. 7-Hydroxymitragynine, Mitragynine, Paynantheine, Speciociliatine, Speciogynine, Ciliaphylline, Corynantheidine, Corynoxine A, Corynoxine B, Isomitraphylline, Isorhynchophylline, Mitraphylline, Rhynchophylline, Speciophylline, Speciofoline, Epicatechin, 7-Hydroxyspecioliatine, 9-Hydroxycorynantheidine, Corynoxeine, Isopteropodine, Isorhynchophylline Oxindole, Tetrahydroalstonine, Mitragynine Oxindole B, and Mitragynine Oxindole A naturally occur in Mitragyna speciosa. Ajmalicine naturally occurs in Catharanthus roseus and Mitragyna speciosa. In nature, 7-Hydroxymitragynine, Mitragynine, Paynantheine, Speciociliatine, Speciogynine, Ajmalicine, Ciliaphylline, Corynantheidine, Corynoxine A, Corynoxine B, Isomitraphylline, Isorhynchophylline, Mitraphylline, Rhynchophylline, Speciophylline, Speciofoline, Epicatechin, 7-Hydroxyspecioliatine, 9-Hydroxycorynantheidine, Corynoxeine, Isopteropodine, Isorhynchophylline Oxindole, Tetrahydroalstonine, Mitragynine Oxindole B, and Mitragynine Oxindole A always occur as a minor component within a mixture of many other molecules.

A pharmaceutical formulation of the disclosure may comprise, consist essentially of, or consist of (a) a purified kratom compound discussed above and (b) a second active compound selected from a serotonergic drug, a purified psilocybin derivative, a purified cannabinoid, or a purified terpene and (c) a pharmaceutically acceptable excipient. The purified kratom compound and the second active compound are each present in a therapeutically effective amount using a purposefully engineered and unnaturally occurring molar ratios.

A pharmaceutical formulation of the disclosure may comprise a composition of the disclosure and a serotonergic drug, a purified psilocybin derivative, a purified cannabinoid, or a purified terpene, each present in a therapeutically effective amount using a purposefully engineered and unnaturally occurring molar ratios. Published US applications US 2018/0221396 A1 and US 2019/0142851 A1 disclose compositions comprising a combination of a purified psilocybin derivative with a second purified psilocybin derivative, with one or two purified cannabinoids or with a purified terpene. The disclosures of US 2018/0221396 A1 and US 2019/0142851 A1 are incorporated herein by reference. According to this disclosure composition containing a purified kratom compound as discussed above may be used in place of a “purified psilocybin derivative” in the compositions described in US 2018/0221396 A1 and US 2019/0142851 A1. Accordingly, the disclosure provides a pharmaceutical formulation comprising as (a) a purified kratom compound according to this disclosure and as a second component selected from (a) a purified psilocybin derivative, (b) one or two purified cannabinoids and (c) a purified terpene; with the rest being at least one suitable pharmaceutical excipient or at least one other adjuvant, as discussed below. Such a composition may be a pharmaceutical composition wherein the components are present individually in therapeutic effective amounts or by combination in a therapeutically effective amount to treat a disease, disorder, or condition as described herein.

A serotonergic drug refers to a compound that binds to, blocks, or otherwise influences (e.g., via an allosteric reaction) activity at a serotonin receptor as described in paragraphs [0245]-[0253] of US 2018/0221396 A1 and [0305]-[0311] US 2019/0142851 A1 as well as the disclosed preferred embodiments, incorporated here by reference. Some exemplary serotonergic drugs include the following molecules: 6-Allyl-N, N-diethyl-NL, N, N-Dibutyl-T, N,N-Diethyl-T, N,N-Diisopropyl-T, 5-Methyoxy-alpha-methyl-T, N, N-Dimethyl-T, 2, alpha-Dimethyl-T, alpha, N-Dimethyl-T, N,N-Dipropyl-T, N-Ethyl-N-isopropyl-T, alpha-Ethyl-T, 6,N,N-Triethyl-NL, 3,4-Dihydro-7-methoxy-1-methyl-C, 7-Methyoxy-1-methyl-C, N,N-Dibutyl-4-hydroxy-T, N,N-Diethyl-4-hydroxy-T, N,N-Diisopropyl-4-hydroxy-T, N,N-Dimethyl-4-hydroxy-T, N, N-Dimethyl-5-hydroxy-T, N, N-Dipropyl-4-hydroxy-T, N-Ethyl-4-hydroxy-N-methyl-T, 4-Hydroxy-N-isopropyl-N-methyl-T, 4-Hydroxy-N-methyl-N-propyl-T, 4-Hydroxy-N,N-tetramethylene-T Ibogaine, N,N-Diethyl-L, N-Butyl-N-methyl-T, N, N-Diisopropyl-4,5-methylenedioxy-T, N,N-Diisopropyl-5,6-methylenedioxy-T, N,N-Dimethyl-4,5-methylenedioxy-T, N,N-Dimethyl-5,6-methylenedioxy-T, N-Isopropyl-N-methyl-5,6-methylenedioxy-T, N,N-Diethyl-2-methyl-T, 2,N,N-Trimethyl-T, N-Acetyl-5-methoxy-T, N,N-Diethyl-5-methoxy-T, N,N-Diisopropyl-5-methoxy-T, 5-Methoxy-N,N-dimethyl-T, N-Isopropyl-4-methoxy-N-methyl-T, N-Isopropyl-5-methoxy-N-methyl-T, 5,6-Dimethoxy-N-isopropyl-N-methyl-T, 5-Methoxy-N-methyl-T, 5-Methoxy-N,N-tetramethylene-T, 6-Methoxy-1-methyl-1,2,3,4-tetrahydro-C, 5-Methoxy-2, N, N-trimethyl-T, N, N-Dimethyl-5-methylthio-T, N-Isopropyl-N-methyl-T, alpha-Methyl-T, N-Ethyl-T, N-Methyl-T, 6-Propyl-N L, N,N-Tetramethylene-T, Tryptamine, and 7-Methoxy-1-methyl-1,2,3,4-tetrahydro-C, alpha, N-Dimethyl-5-methoxy-T. For additional information regarding these compounds See Shulgin, A. T., & Shulgin, A. (2016). Tihkal: The Continuation. Berkeley, Calif.: Transform Press. In one embodiment, a serotonergic drug is chosen from alprazolam, amphetamine, aripiprazole, azapirone, a barbiturate, bromazepam, bupropion, buspirone, a cannabinoid, chlordiazepoxide, citalopram, clonazepam, clorazepate, dextromethorphan, diazepam, duloxetine, escitalopram, fluoxetine, flurazepam, fluvoxamine, lorazepam, lysergic acid diethylamide, lysergamide, 3,4-methylenedioxymethamphetamine, milnacipran, mirtazapine, naratriptan, paroxetine, pethidine, phenethylamine, psicaine, oxazepam, reboxetine, serenic, serotonin, sertraline, temazepam, tramadol, triazolam, a tryptamine, venlafaxine, vortioxetine, and/or derivatives thereof.

Exemplary psilocybin derivatives include but are not limited to psilocybin itself and the psilocybin derivates described in paragraphs [0081]-[0109] of US 2018/0221396 A1 and [082]-[0110] US 2019/0142851 A1 as well as the disclosed preferred embodiments, incorporated here by reference. In one embodiment, the compositions disclosed herein comprise one or more purified psilocybin derivatives chosen from: [3-(2-Dimethylaminoethyl)-1H-indol-4-yl] dihydrogen phosphate, 4-hydroxytryptamine, 4-hydroxy-N,N-dimethyltryptamine, [3-(2-methylaminoethyl)-1H-indol-4-yl] dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl] dihydrogen phosphate, and 4-hydroxy-N,N,N-trimethyltryptamine.

Exemplary cannabinoids include but are not limited to the cannabinoids described in paragraphs [0111]-[0159] of US 2018/0221396 A1 and [0112]-[0160] US 2019/0142851 A1 as well as the disclosed preferred embodiments, incorporated here by reference. Examples of cannabinoids within the context of this disclosure include the following molecules: Cannabichromene (CBC), Cannabichromenic acid (CBCA), Cannabichromevarin (CBCV), Cannabichromevarinic acid (CBCVA), Cannabicyclol (CBL), Cannabicyclolic acid (CBLA), Cannabicyclovarin (CBLV), Cannabidiol (CBD), Cannabidiol monomethylether (CBDM), Cannabidiolic acid (CBDA), Cannabidiorcol (CBD-C1), Cannabidivarin (CBDV), Cannabidivarinic acid (CBDVA), Cannabielsoic acid B (CBEA-B), Cannabielsoin (CBE), Cannabielsoin acid A (CBEA-A), Cannabigerol (CBG), Cannabigerol monomethylether (CBGM), Cannabigerolic acid (CBGA), Cannabigerolic acid monomethylether (CBGAM), Cannabigerovarin (CBGV), Cannabigerovarinic acid (CBGVA), Cannabinodiol (CBND), Cannabinodivarin (CBDV), Cannabinol (CBN), Cannabinol methylether (CBNM), Cannabinol-C2 (CBN-C2), Cannabinol-C4 (CBN-C4), Cannabinolic acid (CBNA), Cannabiorcool (CBN-C1), Cannabivarin (CBV), Cannabitriol (CBT), Cannabitriolvarin (CBTV), 10-Ethoxy-9-hydroxy-delta-6a-tetrahydrocannabinol, Cannbicitran (CBT), Cannabiripsol (CBR), 8,9-Dihydroxy-delta-6a-tetrahydrocannabinol, Delta-8-tetrahydrocannabinol (Δ8-THC), Delta-8-tetrahydrocannabinolic acid (Δ8-THCA), Delta-9-tetrahydrocannabinol (THC), Delta-9-tetrahydrocannabinol-C4 (THC-C4), Delta-9-tetrahydrocannabinolic acid A (THCA-A), Delta-9-tetrahydrocannabinolic acid B (THCA-B), Delta-9-tetrahydrocannabinolic acid-C4 (THCA-C4), Delta-9-tetrahydrocannabiorcol (THC-C1), Delta-9-tetrahydrocannabiorcolic acid (THCA-C1), Delta-9-tetrahydrocannabivarin (THCV), Delta-9-tetrahydrocannabivarinic acid (THCVA), 10-Oxo-delta-6a-tetrahydrocannabinol (OTHC), Cannabichromanon (CBCF), Cannabifuran (CBF), Cannabiglendol, Delta-9-cis-tetrahydrocannabinol (cis-THC), Tryhydroxy-delta-9-tetrahydrocannabinol (triOH-THC), Dehydrocannabifuran (DCBF), and 3,4,5,6-Tetrahydro-7-hydroxy-alpha-alpha-2-trimethyl-9-n-propyl-2,6-metha-no-2H-1-benzoxocin-5-methanol. In one embodiment, the purified cannabinoid is chosen from THC, THCA, THCV, THCVA, CBC, CBCA, CBCV, CBCVA, CBD, CBDA, CBDV, CBDVA, CBG, CBGA, CBGV, or CBGVA.

Exemplary terpenes include but are not limited to the terpenes described in paragraphs [0160]-[0238] of US 2018/0221396 A1 and [0161]-[0300] US 2019/0142851 A1 as well as the disclosed preferred embodiments, incorporated here by reference. In one embodiment, a purified terpene is chosen from acetanisole, acetyl cedrene, anethole, anisole, benzaldehyde, bornyl acetate, borneol, cadinene, cafestol, caffeic acid, camphene, camphor, capsaicin, carene, carotene, carvacrol, carvone, alpha-caryophyllene, beta-caryophyllene, caryophyllene oxide, cedrene, cedrene epoxide, cecanal, cedrol, cembrene, cinnamaldehyde, cinnamic acid, citronellal, citronellol, cymene, eicosane, elemene, estragole, ethyl acetate, ethyl cinnamate, ethyl maltol, eucalyptol/1,8-cineole, eudesmol, eugenol, euphol, farnesene, farnesol, fenchone, geraniol, geranyl acetate, guaia-1 (10),11-diene, guaiacol, guaiol, guaiene, gurjunene, herniarin, hexanaldehyde, hexanoic acid, humulene, ionone, ipsdienol, isoamyl acetate, isoamyl alcohol, isoamyl formate, isoborneol, isomyrcenol, isoprene, isopulegol, isovaleric acid, lavandulol, limonene, gamma-linolenic acid, linalool, longifolene, lycopene, menthol, methyl butyrate, 3-mercapto-2-methylpentanal, beta-mercaptoethanol, mercaptoacetic acid, methyl salicylate, methylbutenol, methyl-2-methylvalerate, methyl thiobutyrate, beta-myrcene, gamma-muurolene, nepetalactone, nerol, nerolidol, neryl acetate, nonanaldehyde, nonanoic acid, ocimene, octanal, octanoic acid, pentyl butyrate, phellandrene, phenylacetaldehyde, phenylacetic acid, phenylethanethiol, phytol, pinene, propanethiol, pristimerin, pulegone, retinol, rutin, sabinene, squalene, taxadiene, terpineol, terpine-4-ol, terpinolene, thujone, thymol, umbelliferone, undecanal, verdoxan, or vanillin. In one embodiment, a purified terpene is chosen from bornyl acetate, alpha-bisabolol, borneol, camphene, camphor, carene, beta-caryophyllene, cedrene, cymene, elemene, eucalyptol, eudesmol, farnesene, fenchol, geraniol, guaiacol, humulene, isoborneol, limonene, linalool, menthol, beta-myrcene, nerolidol, ocimene, phellandrene, phytol, pinene, pulegone, sabinene, terpineol, terpinolene, or valencene.

Disclosed herein are formulations, such as pharmaceutical formulations, utilizing a purposefully chosen composition of a purified kratom compound and a second active compound and optionally an inactive compound, such as an excipient. A pharmaceutical formulation contains a therapeutically effective amount each of a purified kratom compound and a second active compound. A therapeutically effective amount of each of a purified kratom compound and of a second active compound is an amount which correlates to a therapeutic effect and may separately range from, for example, about 0.5-about 200 mg, about 1 mg-about 100 mg, about 2 mg-about 50 mg, about 5 mg-about 25 mg or 25 mg. The actual amount required for treatment of any particular disease, disorder or condition for any particular patient may depend upon a variety of factors including, for example, the particular disease, disorder or condition being treated; the disease state being treated and its severity; the specific pharmaceutical composition employed; the age, body weight, general health, sex and diet of the patient; the mode of administration; the time of administration; the route of administration; and the rate of excretion; the duration of the treatment; any drugs used in combination or coincidental with the specific compound employed; and other such factors well known in the medical arts. The total amount of a purified kratom compound and a second active compound in a formulation may range from about 0.01 to 100 wt. %, from about 0.1 to 100 wt. %, from about 1 to about 99 wt. %, from about 50 to about 90 wt. %, from about 5 to about 75 wt. %, from about 10 to about 50 wt. %, from about 10 to about 25 wt. %, or from about 15 to about 40 wt. %.

A composition of the invention may be formulated in any type or pharmaceutical formulation known in the art. A pharmaceutical formulation of the disclosure may be solid dosage form such as an oral dosage form, e.g. a pill, capsule, and the like, which may or may not be enterically coated. A composition of the disclosure may also be formulated as a pharmaceutical formulation designed to avoid first-pass metabolism. Accordingly, to avoid first pass metabolism, a composition of the invention may be formulated as a transdermal formulation, a sublingual formulation, a buccal formulation, an intravenous (I.V.) formulation, a subcutaneous (S.C.) formulation or an inhalation formulation.

A formulation of a composition of the disclosure may contain additional inactive compounds, such as excipients, binders, stabilizers, permeation enhancers, solubilizers, etc. as known in the art. For example in a pharmaceutical formulation a composition of the disclosure may be admixed with at least one pharmaceutically acceptable excipient such as, for example, sodium citrate or dicalcium phosphate or (a) fillers or extenders, such as, for example, starches, lactose, sucrose, glucose, mannitol, and silicic acid, (b) binders, such as, for example, cellulose derivatives, starch, alginates, gelatin, polyvinylpyrrolidone, sucrose, and gum acacia, (c) humectants, such as, for example, glycerol, (d) disintegrating agents, such as, for example, agar-agar, calcium carbonate, potato or tapioca starch, alginic acid, croscarmellose sodium, complex silicates, and sodium carbonate, (e) solution retarders, such as, for example, paraffin, (f) absorption accelerators, such as, for example, quaternary ammonium compounds, (g) wetting agents, such as, for example, cetyl alcohol, and glycerol monostearate, magnesium stearate and the like (h) adsorbents, such as, for example, kaolin and bentonite, and (i) lubricants, such as, for example, talc, calcium stearate, magnesium stearate, solid polyethylene glycols, sodium lauryl sulfate, or mixtures thereof. In the case of capsules, tablets, and pills, the dosage forms may also comprise buffering agents. Pharmaceutically acceptable adjuvants known in the pharmaceutical formulation art may also be used in the pharmaceutical compositions of the invention. These include, but are not limited to, preserving, wetting, suspending, sweetening, flavoring, perfuming, emulsifying, and dispensing agents.

A pharmaceutical formulation of the disclosure may contain an additional active compound selected from the group consisting of: [2M-H] adipyl arginine, [2M-H] pimeloyl arginine, [2M-H] suberoyl arginine, 1-beta-hydroxybufalin, 1-methyl-2,9-dihydro-1H-pyrido[3-b]indol-6-ol, 11α-hydroxy hellebrigenin, 11α-hydroxymarinobufagin, 11α-hydroxymarinobufagin, 11α-hydroxytelocinobufagin, 11α-hydroxytelo cinobufagin, Marinosin, 11a,19-dihydroxymarinobufagin, 12β-hydroxycinobufagin, 12β-hydroxytetrahydroresibufogenin 3-sulfate,15-hydroxybufalin, 16-desacetyl-19-oxocinobufotalin, 16-desacetylcinobufaginol, 19-hydroxybufalin, 19-hydroxybufalin 3-suberoyl-L-3-methylhistidine ester, 19-hydroxybufalin 3-suberoyl-L-histidine ester, 19,hydroxycinobufotalin, 19-hydroxytelocinobufagin, 19-hydroxytelocinobufagin, 19-oxobufalin, 19-oxocinobufagin, 19-oxocinobufotalin, 19-oxodesacetylcinobufagin, 20,21-epoxyresibufagin, 20R,21-epoxyresibufogenin, 20S,21-epoxyresibufogenin, 1,2,3,4-tetrahydro-6-hydroxy-carboline, 2-methyl-6-hydroxy-1,2,3,4-tetrahydro-β-carboline, 3-((N-azelayl argininyl) marinobufagin), 3-(N-adipoyl argininyl) marinobufagin, 3-(N-azelayl argininyl)-bufalin, 3-(N-dodecadienoyl argininyl) marinobufagin, 3-(N-glutaryl argininyl)-marinobufagin, 3-(N-pimeloyl argininyl) marinobufagin, 3-(N-pimeloyl argininyl) telocinobufagin, 3-(N-sebacyl argininyl) marinobufagin, 3-(N-sebacyl argininyl) telocinobufagin, 3-(N-sebacyl argininyl)-bufalin, 3-(N-suberoyl argininyl) hellebrigenin, 4-amido-3-hydroxymethyl-cyclooctylamidezotetra-alpha-furanone 5-hydroxytryptophan, 5-hydroxy-1H-indole-3-carbaldehyde, 5-hydroxyindoleacetic acid, 5-hydroxytryptophol, 5-methoxytryptophol, 5-methoxyindoleacetic acid, bufothionine, dehydrobufotenine, dehydrobufoteninehydrobromide, O-methylnordehydrobufotenine, 6-hydroxy-1-oxo-3,4-dihydro-β-carboline, Marinobufotoxin, Telocinobufatoxin, Bufalitoxin, 3-(N-undecadienoyl argininyl) marinobufagin, 3-beta-formyloxyresibufogenin, 3-O-formyl-20R,21-epoxy resibufogenin, 3-oxo-20S, 21-epoxyresibufogenin, 4β-hydroxybufalin, 5Z,9Z-3-(1-hydroxybutyl)-5-propylindolizidine, 6-alpha-hydroxycinobufagin, 6Z,10E-4,6-di (pent-4-enyl) quinolizidine, adenine, arenobufagin, arenobufagin 3-sulfate, arenobufagin hemisuberate, arenobufotoxin, argentinogenin, argininesuberoyl marinobufagenin, azelayl arginine, bacagin, BLP1, BLP2, BLP3, bufalin, bufalon, bufotalone, 5-hydroxycinobufaginol, arenobufagin-3-hemisuberate, cinobufotalin-3-hemisuberate, desacetylbufotalin, bufotalin-3-suberoyl-arginineester, bufalin-3-suberoyl-arginineester, cinobufagin-3-suberoyl-arginineester, Resibufogenin-3-suberoyl-arginineester, 11-Hydroxyhellebrigenol, 5-Hydroxygambufotalin, 5,11-Hydroxyresibufaginol, Bufarenogin, 4-Bufarenogin, 5-Hydroxyarenobufagin, Hellebrigenol-3-X1, Hellebrigenol-3-X2, Hellebrigenol-3-X3, 12-Hydroxyresibufogenin, 5-Hydroxybufotalin, 12, oxobufalin, 3-Acetylresibufogenin, 19-oxoresibufagin, 1-Hydroxybufalin, Bufalin-3β-acrylic ester, Telocinobufagin-3-hemisuberate, 3-Oxo-cinobufotalin, Hellebrigenin-3-hemisuberate, 1-Hydroxyarenobufagin, 16-Hydroxytelocinobufagin, Cinobufotalin-3-azelaoyl-arginineester, Cinobufagin-3-succinoyl-arginineester, Bufotalin-3-succinoyl-arginineester, Hellebrigenin-3-suberoyl-arginineester, Bufalin-3-succinoyl-arginineester Telocinobufagin-3-suberoyl-arginineester, Bufotalin-3-pimeloyl-arginineester, 19-Oxocinobufagin-3-adipoyl-arginineester, Telocinobufagin-3-succinoyl-arginineester, Bufalin-3-pim-1, Bufarenogin-3-suberoyl-arginineester, Desacetylcinobufaginol-3-suberoyl-arginineester, Arenobufagin-3-suberoyl-arginineester, Cinobufagin-3-hemisuberate, 12-hydroxybufalin, 16-Hydroxyhellebrigenin, Argentinogenin-3-hemisuberate, Argentinogenin-iso, 3-Oxoargentinogenin, 3-Oxoarenobufagin, Hellebrigenol-3-sulfate, Hellebrigenin-3-sulfate, 19-Hydroxybufalin-3-sulfate, Telocinobufagin-3-sulfate, Arenobufagin-16-acetyl, Resibufogenin-3-formly, 5β,12β-Dihydroxycinobufagin, 16β-Acetoxybufarenogin, 11α, 12β-Dihydroxybufalin, 20S,21-Epoxymarinobufagin, 20S,21-Epoxymarinobufagin-3-Acetyl, Arenobufagin-3-sulfate, Bufalin-3-adipoyl-arginineester, Bufalin-3-pimeloyl-arginineester, Bufotalin-3-sulfate, Cinobufagin-3-adipoyl-arginineester, Cinobufagin-3-glut-2, Cinobufagin-3-pimeloyl-arginineester, Cinobufagin-3-sulfate, Cinobufotalin-3-suberoyl-arginineester, Desacetylcinobufagin-3-succinoyl-arginineester, Gambufotalin-3-adipoyl-arginineester, Gambufotalin-3-pimeloyl-arginineester, Gambufotalin-3-suberoyl-arginineester, Gambufotalin-3-succinoyl-arginineester, Gambufotalin-3-sulfate, Marinobufagin-3-glut-2, Marinobufagin-3-pimeloyl-arginineester, Marinobufagin-3-glut-2, Marinobufagin-3-suberoyl-arginineester, Marinobufagin-3-succinoyl-arginineester, Resibufogenin-3-succinoyl-arginineester, Telocinobufagin-3-glut-2, 12-hydroxycinobufagin, resibufogenin-3-hemisuberate, bufalin-3-hemisuberate, cinobufagin-3-suc-1, Desacetylcinobufagin-3-suc-1, gambufotalin-3-hemisuberate, bufalin 3-adipoyl-L-arginine ester, bufalin 3-pimeloyl-L-arginine ester, bufalin 3-suberoyl-L-histidine ester, bufalin 3-succinoyl-L-arginine ester, bufalin hemisuberate, bufalin-3-sulfate, bufogargarizin A, bufogargarizin B, bufogargarizin C, bufogargarizin D, bufogenin, bufotalin, bufotalin 3-suberoyl-L-1-methylhistidine ester, bufotalin 3-suberoyl-L-3-methylhistidine ester, Vulgarobufotoxin, bufotalin 3-succinoylarginine ester (I), bufotalin 3-sulfate, bufotalinin, bufotalone, bufotoxin, caffeine, cholesterol, cinabufotalitoxin, cinabufotoxin, cinobufagin, cinobufagin 3-adipoyl-L-arginine ester, cinobufagin 3-glutaryl-L-arginine ester, cinobufagin 3-pimeloyl-L-arginine ester, Cinobufotoxin, cinobufagin 3-succinoyl-L-arginine ester, cinobufagin 3-sulfate, cinobufagin hemisuberate, cinobufaginol, cinobufotalin cinobufotalin 3-suberoyl-L-arginine ester, cyclo (pro-gly) dipeptide, desacetylcinobufagin, desacetylcinobufagin 3-hemisuccinate, desacetylcinobufagin 3-succinyl-L-arginine, desacetylcinobufotalin, Daigredorigenin, dopamine, epinephrine, Eritadenine, gamabufotalin, gamabufotalin 3-adipoyl-L-arginine ester, gamabufotalin 3-pimeloyl-L-arginine ester, Gamabufotalitoxin, gamabufotalin 3-succinoyl-L-arginine ester, gamabufotalin 3-sulfate, gamabufotalin hemisuberoate, gamabufotaliniol, gamma sitosterol, guanine, hellebrigenin, Hellebritoxin, hellebrigenol, hypoxanthine, indoleacetic acid, leucine, marinobufagin, marinobufagin 3-glytaryl-L-arginine ester, marinobufagin 3-pimeloyl-L-arginine ester, marinobufagin 3-suberoyl-L-arginine ester, marinobufagin 3-suberoyl-L-glutamine ester, marinobufagin 3-succinoyl-L-arginine ester, marinobufagin 3-sulfate, marinoic acid, morphine, N-(2-(5-hydroxy-1H-indol-3-yl)ethyl)-N-methylformamide, nicotinamide, nicotinic acid, norepinephrine, palmitic acid cholesterol ester, precoccinelline, resibufagin, resibufagin 3-sulfate, resibufaginol, resibufogenin, resibufagenin, resibufogenin 3-suberoyl-L-arginine ester, resibufogenin 3-succinoyl-L-arginine ester, resibufogenin hemisuberate, resibufotoxin, sebacyl arginine, shepherdine, suberic acid, succinic acid, telocinobufagin, Telocinobufogenin, telocinobufagin 3-glutaryl-L-arginine ester, telocinobufagin 3-suberoyl-L-arginine ester, telocinobufagin 3-suberoyl-L-glutamine ester, thiamethoxam, thymine, tricaine methanesulfonate, uracil, valine, Bombinakinin M, Maximin 1, Maximin, 2, Maximin 3, Maximin 4, Maximin 5, Maximin 6, Maximin 7, Maximin 8, Maximin 9, Maximin 10, Maximin H1, Maximin H2 Maximin H3, Maximin H4, Maximin H5, Maximin H6, Maximin H7, Maximin H8, Maximin H9, Maximin H10, Maximin H11, Maximin H12, Maximin H13, Maximin H14, Maximin H15, Maximin H16, Bradykinin, (Thr (6))-bradykinin, Bombinakinin-GAP, sleep-inducing factor (SIF), xanthine and mixtures thereof.

The compositions and formulations disclosed herein are products of human ingenuity, i.e., made by humans and substantially different from how they are found in nature. The disclosed compositions and formulations can be distinguished from naturally occurring forms by comparing the cellular pharmacology of the disclosed formulations with that of naturally occurring forms. The disclosed compositions and formulations can be distinguished from naturally occurring forms by comparing the molar ratios of compounds within the disclosed formulations with those found in nature. The disclosed compositions and formulations can also be distinguished from naturally occurring forms by comparing the molar ratios of compounds within the disclosed formulations with reference compounds that are present alongside the said compounds when those compounds are found in nature.

At the time of this disclosure, kratom compounds were only available within complex mixtures comprising other compounds or matter from their natural sources, i.e. plant matter and other compounds. All data indicate that the presence and amounts of psychoactive compounds within naturally occurring samples are considered highly variable. Plant extracts often do not provide the same physical, cellular, and/or clinical properties as formulations made by combining particular compounds of known purity. In contrast, each of the compositions disclosed herein differs from previously known compositions in significant ways. For example, in the disclosed compositions and formulations, the ratio of kratom compounds (e.g., the purified kratom compound to the second active compound) or the ratio of a kratom compound to a naturally occurring reference compound (e.g., cellulose, ligin, chlorophyll, etc.) differs from those previously disclosed or otherwise naturally occurring. This disclosure provides compositions and formulations made with known amounts of known compounds, including known amounts of kratom compounds. Such formulations allow for administering consistent amounts of kratom compounds, which provides the user or subject with consistent and reliable effects.

A “purified” compound is a compound that is in a pure chemical form, not as it existed in nature. A “purified” compound is a higher purity (% purity) than is found in nature. The compound may be extracted and purified by means known in the art. For example, a kratom compound may have been chromatographed, for example by gas chromatography, liquid chromatography (e.g., LC, HPLC, etc.), flush column chromatography, etc. or subject to crystallization, distillation, or sublimation. A compound may be purified by two or more purifications steps using those techniques or a composition of those techniques. A purified compound is a compound that is 80-100% pure, 90-100% pure or 95-100% pure. Within the context of this disclosure, the term “purified” means separated from other materials, such as plant or fungal material, e.g., protein, chitin, cellulose, or water. A purified compound is substantially free of other materials. For example, a purified compound is substantially free from a second kratom compound; substantially free from histidine; substantially free from a biological material, such as mold, fungus, plant mater, or bacteria; or substantially free from a different unwanted compound, e.g., a compound correlated with unwanted side effects.

The disclosure also provides methods of regulating the activity of a neurotransmitter receptor by administering to a person in need thereof an effective dose of a composition of the disclosure or administering to a person in need thereof a formulation of the disclosure. The methods disclosed herein comprise treating a psychological disorder, e.g., an anxiety disorder, a compulsive disorder (e.g., an addiction), a depressive disorder, etc., with a disclosed composition or a disclosed pharmaceutical formulation. In one embodiment, the methods disclosed herein comprise treating a psychological disorder, e.g., an anxiety disorder, a compulsive disorder (e.g., and addiction), a depressive disorder, etc., by administering to a subject in need of treatment one or more of the compositions disclosed herein and a neurotransmitter activity modulator, e.g., a serotonergic drug, a dopaminergic drug, etc. The psychological disorder may be chosen from depression, psychotic disorder, schizophrenia, schizophreniform disorder (acute schizophrenic episode); schizoaffective disorder; bipolar I disorder (mania, manic disorder, manic-depressive psychosis); bipolar II disorder; major depressive disorder; major depressive disorder with psychotic feature (psychotic depression); delusional disorders (paranoia); Shared Psychotic Disorder (Shared paranoia disorder); Brief Psychotic disorder (Other and Unspecified Reactive Psychosis); Psychotic disorder not otherwise specified (Unspecified Psychosis); paranoid personality disorder; schizoid personality disorder; schizotypal personality disorder; anxiety disorder; social anxiety disorder; substance-induced anxiety disorder; selective mutism; panic disorder; panic attacks; agoraphobia; attention deficit syndrome, post-traumatic stress disorder (PTSD), premenstrual dysphoric disorder (PMDD), and premenstrual syndrome (PMS).

### REFERENCES

1. Tohar N. Volatile compounds and alkaloids from the aqueous extract of Mitragyna speciosa and their in vitro and in vivo anti-inflammatory studies. Doctoral thesis. University of Malaya, Kuala Lumpur. 2016. https://pdfs.semanticscholar.org/6279/b3f059c3225e71b6f0bb36cb1fa3ff0bc810.pdf.
2. Kruegel A C, Grundmann O. The medicinal chemistry and neuropharmacology of kratom: A preliminary discussion of a promising medicinal plant and analysis of its potential for abuse. Neuropharmacology. 2018; 134:108-120. doi: 10.1016/j.neuropharm.2017.08.026
3. Boyer E W, Babu K M, Adkins J E, McCurdy C R, Halpern J H. Self-treatment of opioid withdrawal using kratom (Mitragynia speciosa korth). Addiction. 2008; 103 (6): 1048-1050. doi: 10.1111/j.1360-0443.2008.02209.x
4. Sakaran R, Othman F, Jantan I, Thent Z C, Das S. Effect of Subacute Dose of Mitragyna Speciosa Korth Crude Extract in Female Sprague Dawley Rats. Journal of Medical and Bioengineering. 2014; 3 (2): 98-101. doi: 10.12720/jomb.3.2.98-101
5. Adkins J, Boyer E, McCurdy C. Mitragyna speciosa, A Psychoactive Tree from Southeast Asia with Opioid Activity. Current Topics in Medicinal Chemistry. 2011; 11 (9). doi: info: doi/10.2174/156802611795371305
6. Matsumoto K. Pharmacological Studies on 7-Hydroxymitragynine, Isolated from the Thai Herbal Medicine Mitragyna speciosa: Discovery of an Orally Active Opioid Analgesic. Doctoral Thesis. Chiba University, Chiba, Japan. 2006.
7. Raffa R B, Beckett J R, Brahmbhatt V N, et al. Orally Active Opioid Compounds from a Non-Poppy Source. Journal of Medicinal Chemistry. 2013; 56 (12): 4840-4848. doi: 10.1021/jm400143z
8. Takayama H. Chemistry and Pharmacology of Analgesic Indole Alkaloids from the Rubiaceous Plant, Mitragyna speciosa. Chem Pharm Bull. 2004; 52 (8): 916-928.
9. Takayama H, Ishikawa H, Kurihara M, et al. Studies on the Synthesis and Opioid Agonistic Activities of Mitragynine-Related Indole Alkaloids: Discovery of Opioid Agonists Structurally Different from Other Opioid Ligands. Journal of Medicinal Chemistry. 2002; 45 (9): 1949-1956. doi: 10.1021/jm010576e
10. Hooper D. The anti-opium leaf. Pharmaceutical Journal. 1907; 78:453.
11. Field E. XCVIII.-Mitragynine and mitraversine, two new alkaloids from species of Mitragynia. Journal of the Chemical Society, Transactions. 1921; 119 (0): 887-891. doi: 10.1039/CT9211900887
12. Zacharias D E, Rosenstein R D, Jeffrey G A. The structure of mitragynine hydroiodide. Acta Cryst. 1965; 18 (6): 1039-1043. doi: 10.1107/S0365110X65002499
13. Shellard E. The alkaloids of Mitragyna with special reference to those of Mitragyna speciosa, Korth. Bulletin on Narcotics. 1974; 26 (2): 41-55.
14. Leon F, Habib E, Adkins J E, Furr E B, Mccurdy C R, Cutler S J. Phytochemical Characterization of the Leaves of Mitragyna Speciosa Grown in USA. Natural Product Communications. 2009; 4 (7): 1934578X0900400. doi: 10.1177/1934578X0900400705
15. Kruegel A C, Gassaway M M, Kapoor A, et al. Synthetic and Receptor Signaling Explorations of the Mitragyna Alkaloids: Mitragynine as an Atypical Molecular Framework for Opioid Receptor Modulators. J Am Chem Soc. 2016; 138 (21): 6754-6764. doi: 10.1021/jacs.6b00360
16. Matsumoto K, Horie S, Ishikawa H, et al. Antinociceptive effect of 7-hydroxymitragynine in mice: Discovery of an orally active opioid analgesic from the Thai medicinal herb Mitragyna speciosa. Life Sciences. 2004; 74 (17): 2143-2155. doi: 10.1016/j.lfs.2003.09.054
17. Ponglux D, Wongseripipatana S, Takayama H, et al. A new indole alkaloid, 7α-hydroxy-7H-mitragynine from Mitragyna speciosa in Thailand. Planta Medica. 1994; 60:580-581.
18. Carpenter J M, Criddle C A, Craig H K, et al. Comparative effects of Mitragyna speciosa extract, mitragynine, and opioid agonists on thermal nociception in rats. Fitoterapia. 2016; 109:87-90. doi: 10.1016/j.fitote.2015.12.001
19. Reanmongkol W, Keawpradub N, Sawangjaroen K. Effects of the extracts from Mitragyna speciosa Korth. leaves on analgesic and behavioral activities in experimental animals. J Sci Technol. 2007; 29 (1): 39-48.
20. Sabetghadam A, Ramanathan S, Mansor S M. The evaluation of antinociceptive activity of alkaloid, methanolic, and aqueous extracts of Malaysian Mitragyna speciosa Korth leaves in rats. Pharmacognosy Res. 2010; 2 (3): 181-185. doi: 10.4103/0974-8490.65514
21. Sabetghadam A, Navaratnam V, Mansor S M. Dose-Response Relationship, Acute Toxicity, and Therapeutic Index between the Alkaloid Extract of Mitragyna speciosa and Its Main Active Compound Mitragynine in Mice: Mitragynine Efficacy and Safety. Drug Development Research. 2013; 74 (1): 23-30. doi: 10.1002/ddr.21052
22. Shaik Mossadeq W M, Sulaiman M R, Tengku Mohamad T A, et al. Anti-Inflammatory and Antinociceptive Effects of Mitragyna speciosa Korth Methanolic Extract. Medical Principles and Practice. 2009; 18 (5): 378-384. doi: 10.1159/000226292
23. Macko E, Weisbach J A, Douglas B. Some observations on the pharmacology of mitragynine. Arch Int Pharmacodyn Ther. 1972; 198 (1): 145-161.

1. An engineered and unnatural composition comprising:
(a) a first purified kratom compound; and
(b) a second purified kratom compound,
wherein the first and the second purified kratom compounds have been separated from plant material and are substantially free from non-alkaloid plant matter,
wherein the first and the second purified kratom compounds are present in the composition in a definite, chemically defined, non-natural molar ratio, and
wherein the first and the second purified kratom compounds present in the composition are of known purity and in known amounts, thereby enabling administration of consistent amounts of the first and the second purified kratom compounds to a subject across different portions taken from the same composition.

2. The composition of claim 1, wherein the first purified kratom compound is mitragynine and the second purified kratom compound is 7-hydroxymitragynine.

3. The composition of claim 1, further comprising an excipient.

4. The composition of claim 1, further comprising:
a purified terpene,
wherein the purified terpene is present in the composition in a definite, chemically defined, non-natural molar ratio.

5. The composition of claim 4, wherein the purified terpene is selected from limonene, linalool, eugenol, geraniol, citronellol, pinene, beta-myrcene, carvone, anethole, and menthol.

6. The composition of claim 1, wherein the composition is substantially free from a third kratom compound that is different than the first and the second purified kratom compounds.

7. The composition of claim 6, wherein the third kratom compound that the composition is substantially free from is 7-hydroxymitragynine.

8. The composition of claim 6, wherein the first and the second purified kratom compounds are independently selected from the group consisting of Mitragynine, Paynantheine, Speciociliatine, Speciogynine, Ajmalicine, Ciliaphylline, Corynantheidine, Corynoxine A, Corynoxine B, Isomitraphylline, Isorhynchophylline, Mitraphylline, Rhynchophylline, Speciophylline, Speciofoline, Epicatechin, 7-Hydroxyspecioliatine, 9-Hydroxycorynantheidine, Corynoxeine, Isopteropodine, Isorhynchophylline Oxindole, Tetrahydroalstonine, Mitragynine Oxindole B, Mitragynine Oxindole A, and a salt thereof, and wherein the third kratom compound that the composition is substantially free from is 7-hydoxymitragynine.

9. The composition of claim 1, wherein the first and the second purified kratom compounds are independently selected from the group consisting of 7-Hydroxymitragynine, Mitragynine, Paynantheine, Speciociliatine, Speciogynine, Ajmalicine, Ciliaphylline, Corynantheidine, Corynoxine A, Corynoxine B, Isomitraphylline, Isorhynchophylline, Mitraphylline, Rhynchophylline, Speciophylline, Speciofoline, Epicatechin, 7-Hydroxyspecioliatine, 9-Hydroxycorynantheidine, Corynoxeine, Isopteropodine, Isorhynchophylline Oxindole, Tetrahydroalstonine, Mitragynine Oxindole B, Mitragynine Oxindole A, and a salt thereof.
